SEQUENCE LISTING (15 sequences)

SEQ ID NO: 1 - protein, 98 aa, Homo sp.
MKLSHRIAAMAATAGITVAAFAAPASAMESGKATSYAELCNEYRKLVRNGKLACTRENDPIQGPDGKVHGNTCSMCEVFFQAEEEEKKKKEGESRNKR

SEQ ID NO: 2 - protein, 97 aa, Homo sp.
MKLSHRIAAMAATAGITVAAFAAPASAMEIVKLCSQYQNQAKNGILFCTRENDPIRGPDGKMHGNLCSMCQAYFQAENEEKKKAEARARNSEQIDNO

SEQ ID NO: 3 - protein, 62 aa, Pediococci sp.
MKKIEKLTEKEMANIIGGKYYGNGVTCGKHSCSVDWGKATTCIINNGAMAWATGGHQGNHKC

SEQ ID NO: 4 - protein, 174 aa, Pediococci sp.
MSKKFWSNIFLALGVFLAFAGVATISVSADSSATIESNTSSKIIDGATYEENIRGVIPITLTQYLHKAQTGEKFIVFVGFKECVHCRKFSPVMKQYLQQSQHPIYYLDYGNNGSFSMASQKQITDFYSTFATPMSFMGTPTVALLDNGKVVSMTAGDDTTLSDLQQITADYNNQ

SEQ ID NO: 5 - protein, 724 aa, Pediococci sp.
MWTQKWHKYYTAQVDENDCGLAALNMILKYYGSDYMLAHLRQLAKTTADGTTVLGLVKAAKHLNLNAEAVRADMDALTASQLPLPVIVHVFKKNKLPHYYVVYQVTENDLIIGDPDPTVKTTKISKSQFAKEWTQIAIIIAPTVKYKPIKESRHTLIDLVPLLIKQKRLIGLIITAAAITTLISIAGAYFFQLIIDTYLPHLMTNRLSLVAIGLIVAYAFQAIINYIQSFFTIVLGQRLMIDIVLKYVHHLFDLPMNFFTTRHVGEMTSRFSDASKIIDALGSTTLTLFLDMWILLAVGLFLAYQNINLFLCSLVVVPIYISIVWLFKKTFNRLNQDTMESNAVLNSAIIESLSGIETIKSLTGEATTKKKIDTLFSDLLHKNLAYQKADQGQQAIKAATKLILTIVILWWGTFFVMRHQLSLGQLLTYNALLAYFLTPLENIINLQPKLQAARVANNRLNEVYLVESEFSKSREITALEQLNGDIEVNHVSFNYGYCSNILEDVSLTIPHHQKITIVGMSGSGKTTLAKLLVGFFEPQEQHGEIQINHHNISDISRTILRQYINYVPQEPFIFSGSVLENLLLGSRPGVTQQMIDQACSFAEIKTDIENLPQGYHTRLSESGFNLSGGQKQRLSIARALLSPAQCFIFDESTSNLDTITEHKIVSKLLFMKDKTIIFVAHRLNIASQTDKVVVLDHGKIVEQGSHRQLLNYNGYYARLIHNQE

SEQ ID NO: 6 - protein, 71 aa, Pediococci sp.
MKLSHRIAAMAATAGITVAAFAAPASAKYYGNGVTCGKHSCSVDWGKATTCIINNGAMAWATGGHQGNHKC

SEQ ID NO: 7 - protein, 268 aa, Homo sp.
MKLSHRIAAMAATAGITVAAFAAPASAEVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSGGGGSGGGGSGGGGSDILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 8 - protein, 187 aa, Homo sp.
MKLSHRIAAMAATAGITVAAFAAPASASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 9 - protein, 134 aa, Homo sp.
MKLSHRIAAMAATAGITVAAFAAPASASGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKA

SEQ ID NO: 10 - protein, 126 aa, Homo sp.
MKLSHRIAAMAATAGITVAAFAAPASAMGSSAVTGVPVKGQDTVKGRVPFNGQDPVKGQVSVKGQDKVKAQEPVKGPVSTKPGSCPIILIRCAMLNPPNRCLKDTDCPGIKKCCEGSCGMACFVPQ

SEQ ID NO: 11 - protein, 30 aa, synthetic construct
MKLSHRIAAMAATAGITVAAFAAPASAGHK

SEQ ID NO: 12 - protein, 31 aa, synthetic construct
MKLSHRIAAMAATAGITVAAFAAPASAGEKG

SEQ ID NO: 13 - protein, 31 aa, synthetic construct
MKLSHRIAAMAATAGITVAAFAAPASAPKEK

SEQ ID NO: 14 - protein, 32 aa, synthetic construct
MKLSHRIAAMAATAGITVAAFAAPASAGPRPA

SEQ ID NO: 15 - protein, 32 aa, synthetic construct
MKLSHRIAAMAATAGITVAAFAAPASAYAGFL

(19) United States
(12) Patent Application Publication
(10) Pub. No.: US 2024/0000863 A1
(43) Pub. Date: Jan. 4, 2024

(54) Skin Probiotics

(71) Applicant: The Regents of the University of California, Oakland, CA (US)

(72) Inventors: Amin Zargar, Berkeley, CA (US); Jay D. Keasling, Berkeley, CA (US)

(73) Assignee: The Regents of the University of California, Oakland, CA (US)

(21) Appl. No.: 18/458,139

(22) Filed: Aug. 29, 2023

Related U.S. Application Data
(63) Continuation of application No. PCT/US22/19051, filed on Mar. 5, 2022.
(60) Provisional application No. 63/157,564, filed on Mar. 5, 2021.

(51) Int. Cl. A61K 35/741; A61K 9/00; A61K 38/55; A61K 38/20; A61K 38/16; A61K 47/26; A61P 17/00

(52) U.S. Cl. CPC A61K 35/741; A61K 9/0014; A61K 38/55; A61K 2039/505; A61K 38/164; A61K 47/26; A61P 17/00; A61K 38/2066

(57) ABSTRACT

A topical, skin probiotic formulation comprises a living population of Corynebacterium glutamicum bacteria genetically-engineered to produce a skin-bioactive agent, and a nutrient source for the bacteria.

### CROSS-REFERENCES TO RELATED APPLICATIONS

This application is a continuation of PCT/US22/19051, filed Mar. 5, 2022, which claims priority to U.S. Provisional Application No. 63/157,564, filed Mar. 5, 2021, the disclosures of which is hereby incorporated by reference in its entirety for all purposes.

### STATEMENT OF GOVERNMENT SUPPORT

This invention was made with government support under Grant Number GM125179 awarded by the National Institutes of Health. The government has certain rights in the invention.

### REFERENCE TO SEQUENCE LISTING

The present application is being filed along with a Sequence Listing in electronic format. The Sequence Listing file, entitled B21-024-2US.xml, was created on Aug. 30, 2023, and is 15,682 bytes in size. The information in electronic format of the Sequence Listing is incorporated herein by reference in its entirety.

### INTRODUCTION

Existing topical treatments of the skin suffer from short half-lives and repeated numerous applications is not feasible. Patches that deliver continuous treatment are uncomfortable and impractical for large skin areas. Continuous delivery of peptides, antimicrobial peptides and proteins to treat skin disease and skin aging through skin probiotics could be a transformational improvement in skin diseases, skin care, and health-care more broadly.

WO2015184134 (U.S. Pat. No. 10,702,558) relates to treating skin diseases with engineered, skin commensal microorganisms, like Staphylococcus epidermidia. U.S. Pat. No. 9,234,204 relates to protecting human skin by recombinantly expressing mycosporine-like amino acids in plasmids in skin commensal organisms. U.S. Ser. No. 10/293,007 relates to the the treatment of skin diseases with engineered human skin microorganisms, like Propionibacterium acnes.

While providing sustainable infection, skin flora commensal microbes can also be opportunistic pathogens, and impose engineering constraints for producing and secreting compounds at a therapeutic level.

### SUMMARY OF THE INVENTION

The invention provides engineering Corynebacterium glutamicum to produce beneficial topical agents like cosmetics, neutraceuticals, sunscreens, and therapies on the skin surface. The invention provides use of genetically engineered Corynebacterium glutamicum as a host for the production of beneficial compounds to the skin surface. An auxotrophic C. glutamicum (the auxotrophy provides a guarantee preventing unwanted spread) is applied to the skin surface as part of a fermentation medium that can also provide a moisturizer. The formulation includes sugars and other media components that the engineered C. glutamicum use as a nutrient source. C. glutamicum converts the nutrient source to the production of beneficial compounds such as amino acids, oligopeptides, antimicrobial peptides, and proteins.

C. glutamicum has been engineered for over 40 years with a diverse genetic toolbox already developed. Moreover, the C. glutamicum is non-commensal, and will not be engineered to grow on the lipids and proteins of the skin; rather, our media formulations provide growth nutrient(s).

In an aspect the invention provides a topical, skin probiotic formulation comprising a living population of Corynebacterium glutamicum bacteria genetically-engineered to produce a skin-bioactive agent, and a nutrient source for the bacteria.

In an aspect the invention provides use of an engineered strain of Corynebacterium glutamicum that is specially engineered through adaptive laboratory evolution for better growth and bioproduction in the acidic and lipidic environment of the skin.

In embodiments:
the formulation is disposed on the skin of a person;
the bacteria are producing the agent;
the formulation further comprises a skin moisturizer;
the formulation further comprises a humectant, such as glycerin, hyaluronic acid, and propylene glycol, or an emollient, such as shea butter, cocoa butter, and octyldodecanol, or an occlusive, such as petrolatum, cetyl alcohol (hexadecan-1-ol), and lanolin;
the nutrient source comprises one or more sugars;
the bioactive agent is selected from a cosmetic, anti-aging, anti-oxidant, sun-screen, anti-inflammatory, analgesic, and therapeutic compound;
the bioactive agent is selected from signal peptides, carrier peptides, neurotransmitter inhibitor peptides, and enzyme inhibitor peptides;
the bioactive agent is selected from lysine, arginine, cysteine, histidine, alanine, serine, theronine, isoleucine, aspartic acid, valine, citrulline, GHK-Cu, GSH-Cu, mangenese tripeptide, Peptamide-6, carnosine, N-acetyl carnosine, tripeptide-10-citrulline, palmitoyl-tripeptides, palmitoyl-tetrapeptides, palmitoyl-pentapeptides, acetyl-tetrapeptide, hexapeptide-11, tetrapeptide PKEK, hexapeptide-14, silk protein, aquaporin, alpha interferon, Hsp70, transforming growth factors, rice peptides, and soybean peptides;
the bioactive agent is a serine protease inhibitor, such as LEKTI-D5, LEKTI-D6 for treating Netherton Syndrome; and/or
the bioactive agent is an anti-inflammatory compound, such as IL-10.
the bioactive agent is an anti-microbial peptide such as pediocin and nisin.
the bioactive agent is an antibody fragment, such as single chain variable fragmine anti-tumor necrosis factor alpha

In an aspect the invention provides a method of using a subject formulation comprising applying the formulation to the skin of a person in need thereof under conditions wherein the bacteria produce an effective amount of the agent on the skin over a predetermined time range.

In embodiments:
the time range is 1 to 30, 60 or 90 days, or about 4, 8, 12, 24 or 48 hours; and/or
the method further comprises adjusting the concentration of the nutrient source to adjust bioproduction and colonization time, such as wherein the concentration of glucose and glycerol is varied from 2 to 20% of the moisturizer for increasingly longer colonization times.

The invention encompasses all combinations of the particular embodiments recited herein, as if each combination had been laboriously recited, such as wherein

### BRIEF DESCRIPTION OF THE DRAWINGS

FIG. 1. Safety and feasibility data: 3D in vitro cultures show that C. glutamicum does not cause increased cell death; C. glutamicum can maintain a stable colonization profile; C. glutamicum can produce lysine in cultures.

FIG. 2. Longevity of C. glutamicum in moisturizer. 107 CFUs per mL were added to an emulsified moisturizer and placed at room temperature and periodically measured for active C. glutamicum.

FIG. 3. Safety and efficacy of applying to skin a probiotic moisturizer comprising the soil bacterium C. glutamicum producing glutamate.

FIG. 4. (Left) C. glutamicum was fermented and the supernatant was purified with nickel chromatography to isolate the secreted LEKTI-D6 protein and LEKTI-D5 an SDS page gel was run. (Right) A bioactivity inhibition assay of the KLK5 protein against varying concentrations of the purified LEKTI-D6 protein.

### DESCRIPTION OF PARTICULAR EMBODIMENTS AND DELIVERY METHODS OF THE INVENTION

Unless contraindicated or noted otherwise, in these descriptions and throughout this specification, the terms “a” and “an” mean one or more, the term “or” means and/or. The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

We disclose genetically engineering innocuous bacterium Corynebacterium glutamicum to temporarily populate the skin microbiome and deliver molecules and proteins that treat disease as well as improve cosmetic appearance. While Corynebacterium is one of the three most abundant bacterial genera on human skin, the Generally Recognized as Safe (GRAS) organism C. glutamicum is not native to the skin microbiome. Temporary colonization of C. glutamicum, a well-studied organism for protein production, provides an added safety control feature. This platform for continuous production of enzymes and small molecules to the skin surface is a transformative advance in skin therapies.

While manipulation of the skin microbiome through modulation of native bacteria has been conceived in both academia and industry (MatriSys, Azitra, Xycrobe), we take a radically different approach to skin therapy. 9 While the abundance of Corynebacteria in the skin microbiome portends the compatibility of C. glutamicum, our approach is based on engineering a non-native microbe to temporarily colonize the skin. This is achieved through formulation engineering to manipulate the residence time on the skin, akin to media optimization in traditional fermentations. This approach provides increased safety as C. glutamicum cannot colonize the skin environment long-term, and unlike S. epidermidis or C. acnes, is not associated with any skin diseases. The benefits of this approach include slow, stable, consistent and effective release of therapies, at the level of the target cell in the skin. With adaptive laboratory evolution (ALE), we also use an engineered strain of C. glutamicum especially suited to grow and produce bioproducts in the acidic and lipidic environment of the skin.

### Example 1. Safety and Feasibility: Applying Model Skin a Probiotic Moisturizer Comprising the Soil Bacterium C. glutamicum Producing Lysine

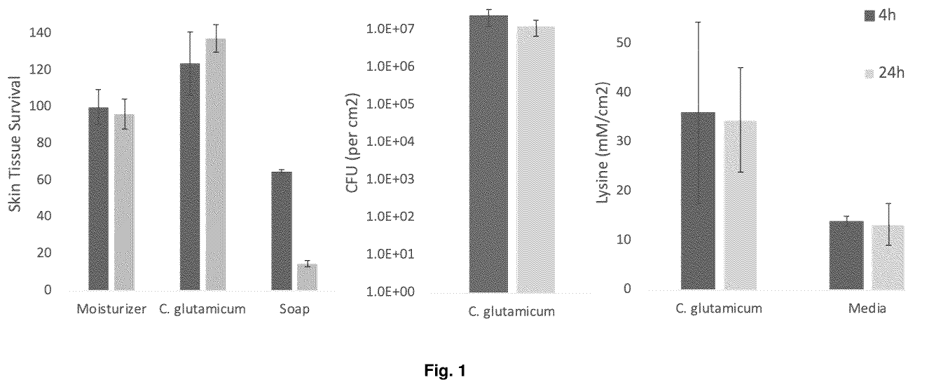

While C. glutamicum is a GRAS (Generally Recognized as Safe), we tested its safety in a human equivalent tissue (EpiDerm). EpiDerm is a highly differentiated 3D tissue model consisting of human-derived epidermal keratinocytes. In a mixture of components common to moisturizers (glycerol, glucose, LB and biotin), we dosed C. glutamicum (˜10⁷ CFUs/cm²) and media alone on the apical surface of EpiDerm. Tissue cell viability assays showed a similar tissue viability, compared to the cell death caused by soap (FIG. 1).

As a proof-of-concept, we dosed the EpiDerm system with a C. glutamicum strain that overproduces lysine, a common moisturizer component. We showed the production of approximately 20 mM more of lysine, thereby illustrating our overall goal of longer effective peptide concentrations through continuous microbial production (FIG. 1).

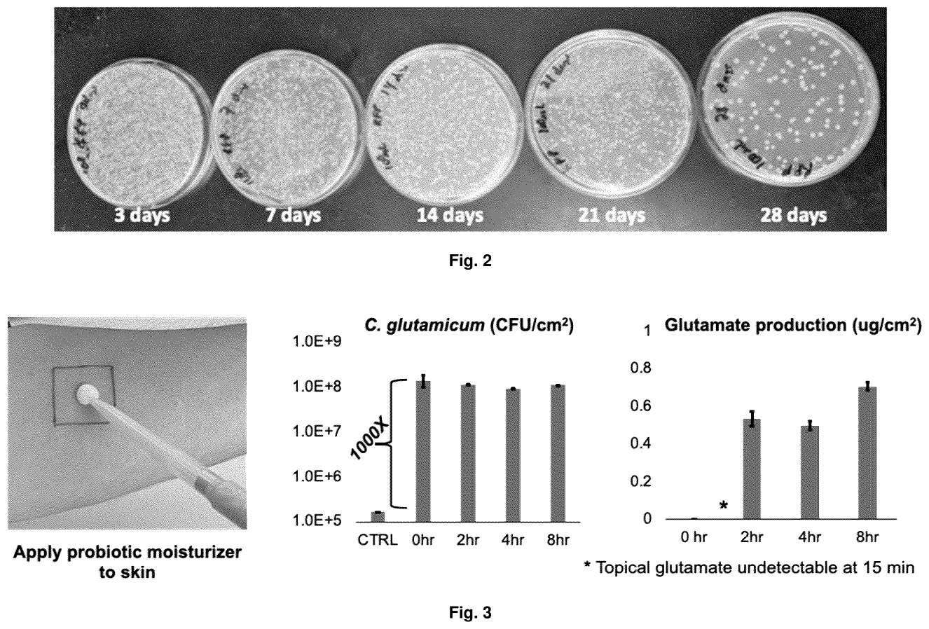

We have also shown that C. glutamicum is active when mixed in with common moisturizer ingredients (water, glycerol, stearic acid, Span60, xanthan gum, dimethicone) in an emulsion for over 28 days (FIG. 2).

Additional examples below demonstrate, inter alia, stable temporary colonization of the human skin by providing nutrients in a moisturizer, the continuous production of glutamate on the skin surface, and the heterologous production of bioactive LEKTI to treat skin diseases such as Netherton Syndrome, atopic dermatitis, psoriasis, and acne rosacea

### Example 2. Safety and Efficacy of Applying to Skin a Probiotic Moisturizer Comprising the Soil Bacterium C. glutamicum Producing Glutamate

Corynebacterium glutamicum is a common soil bacterium and cannot survive on the skin surface. As C. glutamicum consumes sugars and fatty acids, it cannot use the lipids and proteins on the skin surface to produce bioproducts. We have shown that by adding the components for growth to common moisturizer ingredients, we can adjust the environment of the skin where C. glutamicum cannot only stably colonize the skin, but produce bioproducts.

We used common moisturizer ingredients (paraffin oil, water, Span60, dimethicone, and stearic acid) with the needed components for C. glutamicum to grow (glucose, glycerol, yeast extract and biotin) so that C. glutamicum could produce nutrients on the skin surface. The moisturizer is combined in a 1:1 mixture with Corynebacterium glutamicum resuspended at a concentration of 10¹⁰ CFU/mL. Human testing was performed to determine both skin colonization of the soil bacterium and production of glutamate, an amino acid secreted and produced C. glutamicum. We used a control of moisturizer alone to determine local microbiome conditions. After 0, 2, 4, 8 and 24 hours we used a skin swab and determine the colony forming units in a 16 cm² portion on the skin. We determined that C. glutamicum stably colonizes the surface of the skin for 8 hours before dropping back to control conditions after 24 hours. We also found increasing and continuous production of C. glutamicum over 8 hours before dropping back to control conditions after 24 hours. Highlighting the power of C. glutamicum continuous production, a topical glutamate formula of 0.1% glutamate was added to the skin, and was undetectable after 15 minutes likely because of absorption and degradation on the skin surface, demonstrating the utility of bacterial produced compounds continuously on the surface. Our safety data from human trials also demonstrate that applying this organism on human skin is non-irritating and non-allergenic, confirming both safety and efficacy; see, e.g. FIG. 3.

This example uses glutamate, a common moisturizer ingredient, as a proof-of-concept for the production of other therapeutic and aesthetic proteins. Notably, the nutrients in the moisturizer enable the bacterium to colonize the skin, and adjusting the concentration of the nutrient source adjusts the bioproduction and colonization time. We can vary the concentration of glucose and glycerol from 20% of the moisturizer for the longest level of colonization to lacking it completely where the bacterium would die off very quickly.

### Example 3. C. glutamicum Production and Secretion of Bioactive Therapeutic Proteins

Netherton Syndrome is a disease driven by mutations in the SPINK5 gene that encodes lymphoepithelial Kazal-Type-related protease inhibitor (LEKTI) serine protease inhibitors. These mutations cause insufficient inhibition of kallikreins on the surface of the skin, particularly KLK5. Kallikreins are proteases that break down the structural proteins of the epidermis, and insufficient inhibition results in uncontrolled protease activity leading to loss of skin barrier function. Other diseases such as atopic dermatitis, psoriasis, and acne rosacea also have been implicated to have insufficient inhibition of kallikriens by LEKTI.

We engineered C. glutamicum to continuously produce LEKTI proteins, and apply them topically to the lesional skin surface of NS patients, thereby inhibiting KLK5 and restoring the skin surface. The active drug is a modified version LEKTI protein that is known to inhibit KLK5. It has been modified with an N-terminal secretion amino acid signal so that C. glutamicum can secrete the protein out of the cell.

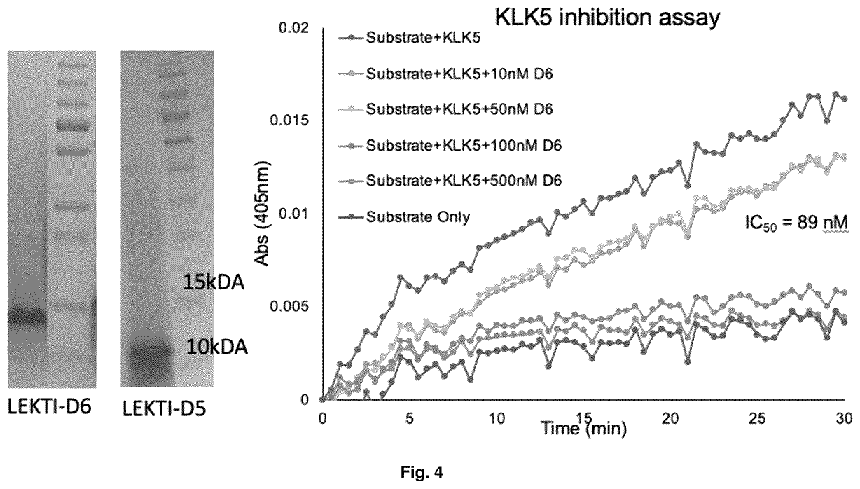

We genetically inserted the LEKTI-D6 and LEKTI-D5 protein into the genome of Corynebacterium glutamicum. The coding sequence is driven from the pH36 promoter and includes an N-terminus secretion tag (porB). The secretion tag improves secretion of the protein into the extracellular environment of C. glutamicum and the secretion tag cleaves the amino acid sequence, resulting in the mature protein secreted to the extracellular environment. The strain was then fermented, and the protein was purified. An SDS page gel of the purified protein shows a clear band at the molecular weights of the encoded proteins (FIG. 4).

LEKTI-D6:
(SEQ ID NO: 01)
MKLSHRIAAMAATAGITVAAFAAPASAMESGKATSYAELCNEYRKLVRNG
KLACTRENDPIQGPDGKVHGNTCSMCEVFFQAEEEEKKKKEGESRNKR
LEKTI-D5:
(SEQ ID NO: 02)
MKLSHRIAAMAATAGITVAAFAAPASAMEIVKLCSQYQNQAKNGILFCTR
ENDPIRGPDGKMHGNLCSMCQAYFQAENEEKKKAEARARN

*Bold font is secretion tag' **Underlined font is active moiety

We showed a correctly folded and bioactive protein by the microbial secretion of LEKTI by C. glutamicum using a KLK5 bioassay. The KLK5 inhibition assay is the foremost method to determine inhibition of KLK5. In the assay, KLK5 is incubated with the substrate Acetyl-YASR-paranitroanilide Uninhibited, KLK5 cleaves the substrate Acetyl-YASR at the arginine position (P4), releasing para-nitroanilide, a chromophore with absorbance 405 nm, resulting in increased absorption at that wavelength. When inhibited, the substrate Acetyl-YASR-paranitroanilide remains intact, and there is no increase in absorption at 405 nm. As a positive control, we used the absence of any inhibitor and the addition of purified Red Fluorescent Protein (RFP). As negative controls, we used a substrate only addition (no KLK5 is added) as well as a 100 uM addition of purified leupeptin, which is known to inhibit KLK5 at high concentrations. Our results showed that RFP and no inhibitor resulted in steadily increasing absorbance over 30 minutes. On the other hand, substrate only or the addition of leupeptin resulted in very little increasing absorbance in that time period. Increasing concentration of LEKTI-D6 showed a dose-dependent quenching of KLK5 with an IC50 of 89 nM.

### Example 4. DNA Cassettes and Validation with Representative Therapeutic Proteins: Antimicrobial Peptides

Continuous production of antimicrobial peptides can treat skin infections and ameliorate bacterial dysbiosis. Current topical approaches often result in recalcitrant re-emergence of the harmful bacteria. By applying C. glutamicum topically, we use competitive inhibition and the continuous production of antimicrobials to permanently eliminate harmful bacteria such as Staphylococcus aureus. Pediocin is a narrow-range antibiotic, and we show that continuous on-site production can be used to treat and prevent S. aureus infections and colonization. In one embodiment, using the native sequence and transporter, we express pediocin as a three gene member family where pedC is the transporter, pedA is the precursor, and pedD cleaves pedA to provide the active pediocin in the supernatant.

Pediocin (Expressed Through 3 Proteins)

>pedA
(SEQ ID NO: 03)
MKKIEKLTEKEMANIIGGKYYGNGVTCGKHSCSVDWGKATTCIINNGAMA
WATGGHQGNHKC
>pedC
(SEQ ID NO: 04)
MSKKFWSNIFLALGVFLAFAGVATISVSADSSATIESNTSSKIIDGATYE
ENIRGVIPITLTQYLHKAQTGEKFIVFVGFKECVHCRKFSPVMKQYLQQS
QHPIYYLDYGNNGSFSMASQKQITDFYSTFATPMSFMGTPTVALLDNGKV
VSMTAGDDTTLSDLQQITADYNNQ
>pedD
(SEQ ID NO: 05)
MWTQKWHKYYTAQVDENDCGLAALNMILKYYGSDYMLAHLRQLAKTTADG
TTVLGLVKAAKHLNLNAEAVRADMDALTASQLPLPVIVHVFKKNKLPHYY
VVYQVTENDLIIGDPDPTVKTTKISKSQFAKEWTQIAIIIAPTVKYKPIK
ESRHTLIDLVPLLIKQKRLIGLIITAAAITTLISIAGAYFFQLIIDTYLP
HLMTNRLSLVAIGLIVAYAFQAIINYIQSFFTIVLGQRLMIDIVLKYVHH
LFDLPMNFFTTRHVGEMTSRFSDASKIIDALGSTTLTLFLDMWILLAVGL
FLAYQNINLFLCSLVVVPIYISIVWLFKKTFNRLNQDTMESNAVLNSAII
ESLSGIETIKSLTGEATTKKKIDTLFSDLLHKNLAYQKADQGQQAIKAAT
KLILTIVILWWGTFFVMRHQLSLGQLLTYNALLAYFLTPLENIINLQPKL
QAARVANNRLNEVYLVESEFSKSREITALEQLNGDIEVNHVSFNYGYCSN
ILEDVSLTIPHHQKITIVGMSGSGKTTLAKLLVGFFEPQEQHGEIQINHH
NISDISRTILRQYINYVPQEPFIFSGSVLENLLLGSRPGVTQQMIDQACS
FAEIKTDIENLPQGYHTRLSESGFNLSGGQKQRLSIARALLSPAQCFIFD
ESTSNLDTITEHKIVSKLLFMKDKTIIFVAHRLNIASQTDKVVVLDHGKI
VEQGSHRQLLNYNGYYARLIHNQE

In another embodiment, we use C. glutamicum secretion tags to secrete the active compound directly.

Pediocin (Expressed Through a Single Protein)

(SEQ ID NO: 06)
MKLSHRIAAMAATAGITVAAFAAPASAKYYGNGVTCGKHSCSVDWGKATT
CIINNGAMAWATGGHQGNHKC

We use the same protocol to deliver alternative antimicrobial peptides, including nisin, a polycyclic antibacterial peptide produced by the bacterium Lactococcus lactis.

### Example 5. Validation with Representative Therapeutic Proteins: Antibodies

We constructed cassettes for on-site production of anti-inflammatory antibodies, antibody fragments, and nanobodies to treat a immunological disorders such as Netherton Syndrome, atopic dermatitis, psoriasis and acne rosacea; the protocols are readily extended to other antibodies targeting IgG, CD20 and other immune targets.

Single Chain Variable Fragment Anti-Tumor Necrosis Factor Alpha

(SEQ ID NO: 07)
MKLSHRIAAMAATAGITVAAFAAPASAEVKLEESGGGLVQPGGSMKLSCV
ASGFIFSNHWMNWVRQSPEKGLEWVAEIRSKSINSATHYAESVKGRFTIS
RDDSKSAVYLQMTDLRTEDTGVYYCSRNYYGSTYDYWGQGTTLTVSGGGG
SGGGGSGGGGSDILLTQSPAILSVSPGERVSFSCRASQFVGSSIHWYQQR
TNGSPRLLIKYASESMSGIPSRFSGSGSGTDFTLSINTVESEDIADYYCQ
QSHSWPFTFGSGTNLEVK

### Example 6. Validation with Representative Therapeutic Proteins: Anti-Inflammatories

We constructed cassettes for on-site production of interleukins, including IL-10, to treat immune hypersensitivity responses on the skin.

IL-10

(SEQ ID NO: 08)
MKLSHRIAAMAATAGITVAAFAAPASASPGQGTQSENSCTHFPGNLPNML
RDLRDAFSRVKTFFQMKDQLDNLLLKESLLEDFKGYLGCQALSEMIQFYL
EEVMPQAENQDPDIKAHVNSLGENLKTLRLRLRRCHRFLPCENKSKAVEQ
VKNAFNKLQEKGIYKAMSEFDIFINYIEAYMTMKIRN

In addition to LEKTI, other protease inhibitors can be used to inhibit skin diseases by continuous production by C. glutamicum, including Secretory leukocyte protease inhibitor and elafin.

Secretory leukocyte protease inhibitor
(SEQ ID NO: 09)
MKLSHRIAAMAATAGITVAAFAAPASASGKSFKAGVCPPKKSAQCLRYKK
PECQSDWQCPGKKRCCPDTCGIKCLDPVDTPNPTRRKPGKCPVTYGQCLM
LNPPNFCEMDGQCKRDLKCCMGMCGKSCVSPVKA
Elafin
(SEQ ID NO: 10)
MKLSHRIAAMAATAGITVAAFAAPASAMGSSAVTGVPVKGQDTVKGRVPF
NGQDPVKGQVSVKGQDKVKAQEPVKGPVSTKPGSCPIILIRCAMLNPPNR
CLKDTDCPGIKKCCEGSCGMACFVPQ*

### Example 7. Validation with Representative Aesthetic Proteins

We also validated the invention with representative short peptide sequences that are beneficial to aesthetics by upregulating the production of collagen or relaxing facial muscles to reduce wrinkling. Continuous production by bacteria greatly increases their residence time, as normally they are absorbed and broken down by the skin. Examples include (encoded aesthetic peptide sequences are underscored):

1. GHK-Cu (complexed with trace copper)
(SEQ ID NO: 11)
MKLSHRIAAMAATAGITVAAFAAPASAGHK
2. GEKG
(SEQ ID NO: 12)
MKLSHRIAAMAATAGITVAAFAAPASAGEKG
3. PKEK
(SEQ ID NO: 13)
MKLSHRIAAMAATAGITVAAFAAPASAPKEK
4. GPRPA
(SEQ ID NO: 14)
MKLSHRIAAMAATAGITVAAFAAPASAGPRPA
5. YAGFL
(SEQ ID NO: 15)
MKLSHRIAAMAATAGITVAAFAAPASAYAGFL

1. A topical, skin probiotic formulation comprising a living population of Corynebacterium glittamicum bacteria genetically-engineered to produce a skin-bioactive agent, and a nutrient source for the bacteria.

2. The formulation of claim 1, disposed on the skin of a person.

3. The formulation of claim 1, wherein the bacteria are producing the agent.

4. The formulation of claim 1, further comprising a skin moisturizer.

5. The formulation of claim 1, further comprising a humectant, such as glycerin, hyaluronic acid, and propylene glycol, or an emollient, such as shea butter, cocoa butter, and octyldodecanol, or an occlusive, such as petrolatum, cetyl alcohol (hexadecan-1-ol), and lanolin.

6. The formulation of claim 1, wherein the nutrient source comprises one or more sugars.

7. The formulation of claim 1, wherein the bioactive agent is selected from a cosmetic, anti-aging, anti-oxidant, sun-screen, anti-inflammatory, antimicrobial, analgesic, and therapeutic compound.

8. The formulation of claim 1, wherein the bioactive agent is selected from signal peptides, carrier peptides, neurotransmitter inhibitor peptides, and enzyme inhibitor peptides.

9. The formulation of claim 1, wherein the bioactive agent is selected from lysine, arginine, cysteine, histidine, alanine, serine, theronine, isoleucine, aspartic acid, valine, citrulline, GHK-Cu, GSH-Cu, mangenese tripeptide, Peptamide-6, carnosine, N-acetyl carnosine, tripeptide-10-citrulline, palmitoyl-tripeptides, palmitoyl-tetrapeptides, palmitoyl-pentapeptides, acetyl-tetrapeptide, hexapeptide-11, tetrapeptide PKEK, hexapeptide-14, silk protein, aquaporin, alpha interferon, Hsp70, transforming growth factors, rice peptides, and soybean peptides.

10. The formulation of claim 1, wherein the bioactive agent is a serine protease inhibitor, such as LEKTI-D6, LEKTI-D5, elafin and secretory leukocyte protease inhibitor for treating Netherton Syndrome, atopic dermatitis, psoriasis, and acne rosacea.

11. The formulation of claim 1, wherein the bioactive agent is a serine protease inhibitor, that is LEKTI-D6, LEKTI-D5, elafin or secretory leukocyte protease inhibitor, for treating Netherton Syndrome, atopic dermatitis, psoriasis, and acne rosacea.

12. The formulation of claim 1, wherein the bioactive agent is an anti-inflammatory compound, such as IL-10 to treat diseases such as Netherton Syndrome, atopic dermatitis, psoriasis, acne rosacea, and chronic wound infection.

13. The formulation of claim 1, wherein the bioactive agent is an anti-inflammatory compound, that is IL-10 to treat diseases such as Netherton Syndrome, atopic dermatitis, psoriasis, acne rosacea, and chronic wound infection.

14. The formulation of claim 1, wherein the bioactive agent is an anti-microbial peptide, such as pediocin and nisin to treat diseases such as Netherton Syndrome, atopic dermatitis, psoriasis, acne rosacea, and chronic wound infection.

15. The formulation of claim 1, wherein the bioactive agent is an anti-microbial peptide, that is pediocin or nisin to treat diseases such as Netherton Syndrome, atopic dermatitis, psoriasis, acne rosacea, and chronic wound infection.

16. The formulation of claim 1, wherein the bioactive agent is an antibody, antibody fragment or nanobody, such as single chain variable fragment anti-tumor necrosis factor alpha to treat diseases such as Netherton Syndrome, atopic dermatitis, psoriasis, acne rosacea, and chronic wound infection.

17. The formulation of claim 1, wherein the bioactive agent is an antibody, antibody fragment or nanobody, that is a single chain variable fragment anti-tumor necrosis factor alpha to treat diseases such as Netherton Syndrome, atopic dermatitis, psoriasis, acne rosacea, and chronic wound infection.

18. A method of using the formulation of claim 1, comprising applying the formulation to the skin of a person in need thereof under conditions wherein the bacteria produce an effective amount of the agent on the skin over a predetermined time range.

19. The method of claim 18 wherein the time range is selected from 4, 8, 12, 24, and 48 hours.

20. The method of claim 18, further comprising adjusting the concentration of the nutrient source to adjust bioproduction and colonization time, such as wherein the concentration of glucose and glycerol is varied from 2 to 20% of the moisturizer for increasingly longer colonization times.
